(19) United States
(12) Patent Application Publication
(10) Pub. No.: US 2007/0281009 A1
(43) Pub. Date: Dec. 6, 2007

(54) N-Acetylglucosamine Tablet Disintegrating In Oral Cavity And Process For Producing The Same

(75) Inventors: Hisayo Kamisono, Shizuoka (JP); Mamoru Okada, Shizuoka (JP); Yoshinari Tagata, Tokyo (JP); Masatami Nakashima, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Yaizu Suisankagaku Industry Co., Ltd., Yaizu-shi (JP)

(21) Appl. No.: 11/722,955

(22) PCT Filed: Dec. 26, 2005
(86) PCT No.: PCT/JP05/23754

§ 371 Date: Jun. 27, 2007

(30) Foreign Application Priority Data
Dec. 27, 2004 (JP) 2004-375802
Mar. 24, 2005 (JP) 2005-085268

(51) Int. Cl. A61K 31/70; A61K 9/20; A61P 19/02

(52) U.S. Cl. 424/464; 514/62

(57) ABSTRACT

It is intended to provide an N-acetylglucosamine tablet, with which N-acetylglucosamine can be taken in an amount at which a physiological activity can be expected in a small number of tablets, and which has an excellent disintegrating property and solubility in the oral cavity and has an adequate hardness to such an extent that it is not difficult to handle; and a process for producing the same. A saccharide having low moldability is mixed with or sprayed on N-acetylglucosamine to perform coating and/or granulation, and a saccharide having high moldability is mixed with the obtained granulated matter to perform molding, whereby the N-acetylglucosamine tablet disintegrating in oral cavity which has a tablet hardness of 5 to 20 kgf and a mass per tablet of 1,000 to 3,000 mg, and which rapidly disintegrates and dissolves in the oral cavity is obtained. The N-acetylglucosamine tablet disintegrating in oral cavity preferably contains 30 to 90% by mass of N-acetylglucosamine, and preferably contains 500 to 2,000 mg of N-acetylglucosamine per tablet.

### TECHNICAL FIELD

The present invention relates to a tablet containing N-acetylglucosamine and a process for producing the same, and specifically to an N-acetylglucosamine tablet disintegrating in oral cavity, which has excellent properties to handle and rapidly disintegrates and dissolves in the oral cavity, and to a process for producing the same.

### BACKGROUND ART

Naturally occurring N-acetylglucosamine is contained in cell walls of crustacea such as shrimp and crab, insects such as beetle and cricket, and fungus, and is one of monosaccharides that are widely present in nature as a constituent unit of chitin. N-acetylglucosamine has about half sweetness of sugar, and is known as a material for synthesizing mucopolysaccharides in a living body, and has physiological activities for beauty, alleviation of arthropathy or improvement memory learning ability, etc. Therefore, it has recently attracted attention as a material of functional foods.

For example, Patent Document 1 below discloses an elastase inhibitor containing at least one selected from glucosamine, glucosamine derivatives, or pharmaceutically acceptable salts thereof, and illustrates N-acetylglucosamine as one example of the glucosamine derivatives.

Patent Document 2 below discloses a memory learning ability-improving agent characterized by including a composition for oral ingestion containing at least one selected from glucosamine or salt thereof, and N-acetylglucosamine as an active ingredient.

Patent Document 3 below discloses an agent of anti-arthropathy including an amino sugar and trehalose as active ingredients, and illustrates N-acetylglucosamine as one example of the amino sugar.

Patent Document 4 below discloses a nutrient auxiliary composition for promoting articular cartilage regeneration and inhibiting cartilage reduction, obtainable by formulating soybean-derived isoflavone and a soybean extract containing isoflavones to an amino sugar containing glucosamine, glucosamine salt, N-acetylglucosamine, and N-acetylglucosamine salt which are obtained by using chitin and chitin hydrolysates as main materials, an amino sugar containing one glucosamines, or an amino sugar composition obtained by selectively mixing these glucosamines.

Patent Document 5 below discloses a food composition characterized by including an amino acid, hyaluronic acid, and amino sugar, and illustrates N-acetylglucosamine as one example of the amino sugar.

Patent Document 6 below discloses a skin care agent containing N-acetylglucosamine as an active ingredient.

Patent Document 7 below discloses a beauty food composition characterized by including one or more amino sugars or derivatives thereof, and illustrates N-acetylglucosamine as one example of the amino sugar derivatives.

Patent Document 8 below discloses an N-acetylglucosamine formulation capable of being used in oral cavity, which is used for treating degenerative and inflammatory diseases in connective tissues and supporting tissues of joint, and associated diseases.

Patent Document 1: JP 2004-83432 A

Patent Document 2: JP 2004-75618 A

Patent Document 3: JP 2002-193811 A

Patent Document 4: JP 2002-3382 A

Patent Document 5: JP 2001-231503 A

Patent Document 6: JP 2001-48789 A

Patent Document 7: JP 2000-50842 A

Patent Document 8: JP Hei 7-103033

### DISCLOSURE OF THE INVENTION

### Problems to be solved by the Invention

The above-mentioned Patent Documents 1 to 8 disclose N-acetylglucosamine-containing compositions in forms of tablets. A tablet has excellent storage stability and portability and can be ingested easily only with water any time and anywhere, and therefore is one of product forms most used for foods or pharmaceuticals.

However, there is presupposition that the conventional N-acetylglucosamine-containing tablet is provided to swallow with water without disintegrating in oral cavity, and the size of one tablet is limited. Therefore, it is necessary to ingest a plurality of N-acetylglucosamine-containing tablets in order to ingest N-acetylglucosamine in an effective amount for providing a physiological activity.

For example, oral administration of 1,000 mg/day of N-acetylglucosamine to human has been confirmed to be effective for enhancing skin beauty (0. Kajimoto et al, J. New Rem. & Clin., 52(3), 71-80, 2000). Meanwhile, ingestion of 500 mg/day or 1,000 mg/day of N-acetylglucosamine dissolved in milk to human has been confirmed to provide an effect of ameliorating arthropathy (O. Kajimoto et al, J. New Rem. & Clin., 49(5), 71-82, 2003).

N-acetylglucosamine-containing tablets described in Patent Documents 1, 4 to 6, and 8 contains N-acetylglucosamine in amounts of 72 mg to 200 mg per tablet (weight: 110 mg to 500 mg). In a case where such tablets are used to provide an adequate physiological activity of N-acetylglucosamine, it is required to continuously ingest many tablets (at least 3 tablets, up to 14 tablets), which is very troublesome and results in difficulty in ingestion.

In the case of a large tablet like as an N-acetylglucosamine-containing tablet described in Patent Document 7 above, which has a weight of 1,000 mg per tablet and contains 500 mg of N-acetylglucosamine per tablet, it is required not only that the tablet be disintegrated in oral cavity but also that the shape of the tablet be maintained while distributing product or carrying, or at the time of ingestion. Therefore, hardness, disintegrating property and the like of the tablet are important but are not disclosed in the patent.

Accordingly, an object of the present invention is to provide: an N-acetylglucosamine tablet, with which N-acetylglucosamine can be ingested in an amount at which a physiological activity can be expected via a small number of tablets, and which has an excellent disintegrating property and solubility in oral cavity, and has an adequate hardness, so that it does not have difficulty in handling; and a process for producing the same.

### Means for Solving the Problems

To attain the above-mentioned objects, according to an aspect of the present invention, an N-acetylglucosamine tablet disintegrating in oral cavity includes N-acetylglucosamine: in which the tablet has a tablet hardness of 5 kgf to 20 kgf and a mass per tablet of 1,000 mg to 3,000 mg, and in which the tablet rapidly disintegrates and dissolves in the oral cavity.

An N-acetylglucosamine tablet of the present invention is large but has disintegrating property to such an extent that normal chewing disintegrates it and has excellent disintegrating property and solubility in oral cavity so that it can be swallowed with saliva without water. Further, the tablet has sufficient hardness to such an extent that it is not difficult to handle, and therefore it does not disintegrate while distributing product, carrying, or at the time of ingestion.

An N-acetylglucosamine tablet disintegrating in oral cavity according to the present invention preferably contains 30% by mass to 90% by mass of N-acetylglucosamine.

Preferably, the tablet contains 500 mg to 2,000 mg of N-acetylglucosamine per tablet.

According to these aspects, one tablet contains a sufficient amount of N-acetylglucosamine, and therefore N-acetylglucosamine can be ingested in an amount at which a physiological activity can be expected via a small number (one, or at most two) of tablets.

Further, the N-acetylglucosamine tablet disintegrating in oral cavity according to the present invention preferably contains a saccharide having low moldability and a saccharide having high moldability, in which the saccharide having low moldability is at least one selected from the group consisting of xylitol, mannitol, lactose, glucose, sucrose, dextrin, sucrose, fructose, xylose, lactulose, fructo-oligosaccharide, malto-oligosaccharide, galacto-oligosaccharide, erythritol, and lactitol, and in which the saccharide having high moldability is at least one selected from the group consisting of maltitol, maltose, sorbitol, and reduced palatinose.

Preferably, the N-acetylglucosamine tablet disintegrating in oral cavity, further contains β-carotene and/or vitamin A, in which an amount of the β-carotene is 0.004% by mass to 0.4% by mass and an amount of vitamin A is 0.0007% by mass to 0.07% by mass.

According to this aspect, an N-acetylglucosamine tablet disintegrating in oral cavity, which has more excellent quality stability, texture, and taste, can be produced. Further, the tablet is expected to provide a synergistic effect of N-acetylglucosamine and β-carotene or vitamin A on physiological functions such as beauty and alleviation of arthropathy.

Moreover, the tablet is preferably packed in a PTP (Press Through Package). According to this aspect, an N-acetylglucosamine tablet having excellent portability and storage stability can be provided.

According to another aspect of the present invention, there is provided a process for producing an N-acetylglucosamine tablet disintegrating in oral cavity, which rapidly disintegrates and dissolves in the oral cavity, characterized by including: a granulation step of mixing or spraying a saccharide having low moldability with or on N-acetylglucosamine to perform coating and/or granulation and a tablet compression step of mixing the granulated matter obtained in the granulation step with a saccharide having high moldability to perform compression.

In the process for producing an N-acetylglucosamine tablet disintegrating in oral cavity according to the present invention, in which the tablet preferably has a tablet hardness of 5 kgf to 20 kgf and a mass per tablet of 1,000 mg to 3,000 mg.

In addition, the tablet preferably contains 30 to 90% by mass of N-acetylglucosamine, 0.01% by mass to 49% by mass of the saccharide having low moldability, and 0.01% by mass to 49% by mass of the saccharide having high moldability.

Further, the tablet preferably contains 500 mg to 2,000 mg of N-acetylglucosamine per tablet.

Still further, β-carotene and/or vitamin A is preferably mixed in the granulation step or tablet compression step.

According to a production process of the present invention, there can be produced an N-acetylglucosamine tablet disintegrating in oral cavity, which contains a sufficient amount of N-acetylglucosamine per tablet, and is large but has excellent disintegrating property and solubility in oral cavity, and has sufficient hardness to such an extent that it is not difficult to handle.

### EFFECT OF THE INVENTION

According to the present invention, there can be provided an N-acetylglucosamine tablet disintegrating in oral cavity, which contains a large amount of N-acetylglucosamine per tablet, with which N-acetylglucosamine can be ingested in an amount at which a physiological activity can be expected via a small number of tablets. An N-acetylglucosamine tablet disintegrating in oral cavity of the present invention has disintegrating property to such an extent that normal chewing disintegrates it. Because of the excellent disintegrating property and solubility in oral cavity, it can be swallowed with saliva without water. Therefore, even if it is a large tablet, one can easily ingest without water having little trouble or difficulty associated in continuous ingestion. Further, the tablet has sufficient hardness to such an extent that it is not difficult to handle. Therefore it has little chance to disintegrate during carrying or at the time of ingestion, so that one can ingest the tablet easily at any place.

### BEST MODE FOR CARRYING OUT THE INVENTION

An N-acetylglucosamine tablet disintegrating in oral cavity of the present invention is intended to ingest by being chewed into two or more pieces in oral cavity and swallowed. It has disintegrating property to such an extent that normal chewing disintegrates it and solubility in oral cavity to such an extent that it can be swallowed with saliva without water.

In the present invention, the phrase “rapidly disintegrating and dissolving in the oral cavity” means that the duration of time, which a healthy adult requires to place and chew one tablet in the oral cavity, then to dissolve the tablet only with saliva in the mouth, and then to almost completely disintegrate the tablet (oral cavity disintegration time), is preferably 30 to 90 seconds, more preferably 20 to 75 seconds, further more preferably 10 to 60 seconds. Specifically, the duration of time can be defined based on statistically significant average value in monitoring evaluation performed by a predetermined number of people.

The source of N-acetylglucosamine used in the present invention is not particularly limited, but it is preferable to use naturally occurred N-acetylglucosamine obtainable from chitin, which is derived from crustacea such as shrimp and crab as a material in accordance with methods described in JP 05-33037 B and JP 2000-281696 A.

That is, the N-acetylglucosamine can be obtained by allowing an enzyme having an ability to hydrolyze N-acetylchito-oligosaccharide (such as lysozyme, chitinase, or chitobiase) to react with a mixture containing N-acetylchito-oligosaccharide obtained by partially hydrolyzing a polysaccharide chitin prepared from shell of crustacea such as shrimp and crab with an acid, followed by purification, if needed.

The thus-obtained N-acetylglucosamine is naturally occurred type that is obtainable without being subjected to chemical synthesis and therefore can be safely ingested as a food. Note that the naturally occurred-type N-acetylglucosamine produced as above is commercially available, and for example, N-acetylglucosamine with a product name of “Marine Sweet” (manufactured by Yaizu Suisankagaku Industry Co., Ltd.), etc. can be used.

In the present invention, highly-pure N-acetylglucosamine obtained by purification may be used, and a mixture of N-acetylglucosamine and chitinoligosaccharide may be used. For example, the mixture of N-acetylglucosamine and chitinoligosaccharide can be obtained as follows.

That is, chitin is partially hydrolyzed with hydrochloric acid, and the hydrolysate solution is neutralized, followed by a desalting treatment by ion-exchange membrane electrodialysis. After the desalting treatment, glucosamine hydrochloride present in the solution is adsorbed and removed using an ion-exchange resin. Then, resultant solution is subjected to enzymatic decomposition to release N-acetylglucosamine. Enzymes in the thus-obtained reaction solution is deactivated, and if needed, an excipient such as dextrin is added, followed by spray drying using spray drier.

The thus-obtained mixture of N-acetylglucosamine and chitinoligosaccharide preferably contains 80% by mass to 99% by mass of N-acetylglucosamine and 1% by mass to 20% by mass of chitinoligosaccharide. Examples of such mixture of N-acetylglucosamine and chitinoligosaccharide include a commercially available mixture of N-acetylglucosamine and chitinoligosaccharide with a product name of “Marine Sweet 40” (manufactured by Yaizu Suisankagaku Industry Co., Ltd.), etc.

An N-acetylglucosamine tablet disintegrating in oral cavity of the present invention preferably further contains a saccharide having low moldability and a saccharide having high moldability.

In the present invention, the saccharide having low moldability refers to a saccharide having a tablet hardness of less than 2 kgf, more preferably less than 1.8 kgf, furthermore preferably less than 1.5 kgf in a case where 200 mg of the saccharide is subjected to tablet compression using a pestle (8 mmφ) at a tableting pressure of 200 kg. Examples of such saccharide include: xylitol, mannitol, lactose, glucose, sucrose, dextrin, sucrose, fructose, xylose, lactulose, fructo-oligosaccharide, malto-oligosaccharide, galacto-oligosaccharide, erythritol, and lactitol; preferably xylitol, mannitol, lactose, glucose, sucrose, and dextrin; particularly preferably dextrin.

The saccharide having high moldability refers to a saccharide having a tablet hardness of 2 kgf or more, more preferably 1.8 kgf or more, further more preferably 1.5 kgf or more in the case where 200 mg of the saccharide is subjected to tablet compression using a pestle (8 mmφ) at a tableting pressure of 200 kg. Examples of such saccharide include: maltitol, maltose, sorbitol, and reduced palatinose; preferably include maltitol.

Moreover, an N-acetylglucosamine tablet disintegrating in oral cavity of the present invention preferably includes β-carotene and/or vitamin A.

Commercially available β-carotene and vitamin A for foods may be used in the tablet. Examples of the commercially-available β-carotene include β-carotene with a product name of “β-carotene 1% cold water soluble powder” (manufactured by DSM Nutrition Japan K.K.), and β-carotene with a product name of “fermented β-carotene 1% powder” (manufactured by Sankyo Lifetech Co., Ltd.), and examples of the commercially-available vitamin A include vitamin A with a product name of “Riken Dry A-S200PT” (manufactured by Riken Vitamin Co., Ltd.) and vitamin A with a product name of “Dry Vitamin A Sankyo” (manufactured by Sankyo Lifetech Co., Ltd.). Note that β-carotene is known to have an ability to promote synthesis of hyaluronic acid (T. Sato et al, Skin Pharmacol Physiol 17, 77-83, 2004) and is expected to provide a synergistic effect with N-acetylglucosamine on physiological functions for beauty, alleviation of arthropathy, etc., and therefore physiological functions of N-acetylglucosamine can be more effectively obtained.

In an N-acetylglucosamine tablet disintegrating in oral cavity of the present invention, the tablet hardness is required to be 5 kgf to 20 kgf and the mass per tablet is required to be 1,000 mg to 3,000 mg in order to contain a sufficient amount of N-acetylglucosamine as well as to provide good texture.

That is, in a case where the tablet hardness is lower than the above-mentioned range, the tablet disintegrates during product distribution and storage, which is not preferable. In the case where the tablet hardness is higher than the above-mentioned range, disintegrating property in oral cavity at the time of ingestion becomes worse, resulting in a tablet with bad texture, which is also not preferable. If the mass per tablet is smaller than the above-mentioned range, it is difficult to mix a sufficient amount of N-acetylglucosamine in one tablet, which is not preferable. If the mass per tablet is larger than the above-mentioned range, quality stability is lowered due to lowered stability in tableting processing, resulting in difficulty in ingesting one tablet in one mouthful, which is also not preferable.

In order to provide a tablet containing a sufficient amount of N-acetylglucosamine per tablet and having good texture, the tablet hardness is preferably 5 kgf to 13 kgf, more preferably 7 kgf to 9 kgf. The mass per tablet is preferably 1,200 mg to 2,000 mg.

In the present invention, the tablet contains preferably 30% by mass to 90% by mass, more preferably 40% by mass to 80% by mass, particularly preferably 50% by mass to 70% by mass of N-acetylglucosamine. If the N-acetylglucosamine content is lower than the above-mentioned range, it is difficult to mix a sufficient amount of N-acetylglucosamine in one tablet, which is not preferable. If the N-acetylglucosamine content is higher than the above-mentioned range, processability for tableting as well as texture becomes worse, which is also not preferable. Specifically, the tablet contains N-acetylglucosamine in an amount of preferably 500 mg to 2,000 mg per tablet, more preferably 700 mg to 1,500 mg per tablet.

Further, the tablet contains preferably 0.01% by mass to 49% by mass, more preferably 0.05% by mass to 20% by mass, particularly preferably 0.1% by mass to 10% by mass of the saccharide having low moldability. If the concentration of the saccharide having low moldability is lower than the above-mentioned range, processability for tableting becomes worse due to insufficient flowability of N-acetylglucosamine in tableting processing, which is not preferable. If the concentration is higher than the above-mentioned range, the tablet hardness becomes excessively high, which is also not preferable.

Moreover, the tablet contains preferably 0.01% by mass to 49% by mass, more preferably 1% by mass to 45% by mass, particularly preferably 20% by mass to 40% by mass of the saccharide having high moldability. If the concentration of the saccharide having high moldability is lower than the above-mentioned range, the tablet has an insufficient hardness, which is not preferable. If the concentration is higher than the above-mentioned range, the tablet hardness of the tablet becomes excessively high, which is also not preferable.

Moreover, in the present invention, the tablet preferably contains 0.004% by mass to 0.4% by mass of β-carotene and 0.007% by mass to 0.07% by mass of vitamin A, and the tablet more preferably contains 0.01% by mass to 0.1% by mass of β-carotene and 0.002% by mass to 0.02% by mass of vitamin A. If the concentrations of β-carotene and vitamin A are lower than the above-mentioned ranges, the tablet provides an insufficient synergistic effect with N-acetylglucosamine on physiological functions for beauty, alleviation of arthropathy, etc., which is not preferable. If the concentrations are higher than the above-mentioned ranges, the amounts of such ingredients exceed standard intake levels, which is also not preferable.

An N-acetylglucosamine tablet disintegrating in oral cavity of the present invention may contain not only the above-mentioned basic ingredients but also another ingredient in an amount within a range which does not provide an adverse effect, and for example, the tablet may appropriately contain: an amino acid such as arginine, taurine, glutamic acid, histidine, or branched amino acid (leucine, isoleucine, valine); an imidazole compound such as histidine, 1-methylhistidine, 3-methylhistidine, anserine, carnosine, homocarnosine, or valenine; octacosanol, citric acid, acetic acid, chitin dimer, chitin pentamer, chitosan hexamer, oligoglucosamine, eicosapentaenoic acid, docosapentaenoic acid, docosahexaenoic acid, chili pepper, ginseng, yeast zinc, or yeast selenium. When such ingredients are mixed in the tablet, various physiological functions can be imparted. The tablet is intended to ingest by being chewed in oral cavity and swallowed. Therefore, by appropriately mixing a taste-imparting ingredient in a tablet from a viewpoint of taste, the tablet can be easily ingested.

Further, an N-acetylglucosamine tablet disintegrating in oral cavity of the present invention is preferably packed with a PTP (Press Through Package). This can improve portability and storage stability and making it easier to take out from the package.

Hereinafter, a process for producing an N-acetylglucosamine tablet disintegrating in oral cavity of the present invention will be described.

(1) Granulation Step

First, a predetermined amount of the saccharide having low moldability was mixed in or sprayed on a predetermined amount of N-acetylglucosamine to perform coating and/or granulation. The coating/granulation process is not particularly limited and may be a known process, and for example, coating/granulation can be performed using a generally used fluid bed granulator, tumbling granulator, etc. Note that processing conditions differ depending on the used apparatus and therefore may be determined case by case.

(2) Tablet Compression Step

The granulated matter obtained in the above-mentioned step and a predetermined amount of the saccharide having high moldability were mixed and molded. The tablet compression process is not particularly limited and may be a known process, and for example, a high-speed rotation tableting machine may be used. The processing conditions were adjusted so as to produce a tablet with a tablet hardness of 5 kgf to 20 kgf, preferably 5 to 13 kgf, more preferably 7 kgf to 9 kgf and with a mass per tablet of 1,000 mg to 3,000 mg, more preferably 1,200 mg to 2,000 mg. One tablet preferably contains 500 mg to 2,000 mg of N-acetylglucosamine.

Note that β-carotene and/or vitamin A is preferably mixed in the granulation step or the tablet compression step.

In the present invention, in the granulation step and/or the tablet compression step, if necessary, a sub material may be used in an amount within a range which does not provide an adverse effect on taste of a tablet and physical properties, and examples thereof include (1) an excipient (saccharide): starch such as starch or dextrin or a starch-degradation product; a polysaccharide such as carrageenan, agar, alginic acid, guar gum, chitosan, xanthan gum; a monosaccharide or a disaccharide such as sugar, glucose, lactose, or maltose; an oligosaccharide such as fructo-oligosaccharide, malto-oligosaccharide, isomalto-oligosaccharide, galacto-oligosaccharide, chitin-oligosaccharide, or chitosan-oligosaccharide; a sugar alcohol such as maltitol, sorbitol, xylitol, or erythritol; etc. (2) a thickener: guar gum, xanthan gum, locust bean gum, carrageenan, alginic acid, pectin, etc. (3) a lubricant (emulsifier): sucrose fatty acid ester, magnesium stearate, calcium stearate, etc.

The above-mentioned excipient (saccharide) may be used at a concentration in the same range as that of the saccharide having low moldability or saccharide having high moldability depending on its moldability. The thickener may be subjected to powder mixing with a major ingredient, followed by granulation processing, or part or whole of the thickener may be dissolved in liquid such as water or ethanol, and sprayed to a major ingredient to perform granulation processing. The thickener may be mixed in the tablet compression step without granulation processing. The lubricant may be mixed in the tablet compression step.

The thus-obtained N-acetylglucosamine tablet disintegrating in oral cavity is preferably packed with a PTP (Press Through Package) in order to improve portability or storage stability as well as to make it easier to take out from the package.

### EXAMPLES

Materials for granulation processing having a composition shown in Table 1 were used to perform granulation processing and drying using a flow granulation apparatus (product name “FD-WH(G)-60”, manufactured by POWREX Corporation) in accordance with a conventional method. Drying was performed at a temperature of 80° C. until the water content reached 1% or less.

After drying, materials for tableting processing having a composition shown in Table 1 were mixed, and tableting processing was carried out in accordance with a conventional method using a tableting machine (product name “TEGA 1024SS4-HY”, manufactured by Kikusui Seisakusyo Ltd.) at a tableting pressure of 1,000 kg, to thereby yield an N-acetylglucosamine tablet disintegrating in oral cavity (diameter: 20 mm, 1,850 mg/tablet).

TABLE 1

| | | Comparative | Comparative | Comparative | Comparative |
|---|---|---|---|---|---|
| Materials | Example | Example 1 | Example 2 | Example 3 | Example 4 |
| (For granulation processing) | | | | | |
| N-acetylglucosamine | 50.0 kg | 50.0 kg | 50.0 kg | 50.0 kg | 50.0 kg |
| 1% β-carotene | 1.8 kg | 1.8 kg | 1.8 kg | 1.8 kg | 1.8 kg |
| 6% vitamin A | 0.1 kg | 0.1 kg | 0.1 kg | 0.1 kg | 0.1 kg |
| Guar gum | 0.1 kg | 0.1 kg | 0.1 kg | 0.1 kg | 0.1 kg |
| Dextrin | 5.0 kg | — | 5.0 kg | — | 5.0 kg |
| Maltitol | — | 27.5 kg | 27.5 kg | 5.0 kg | — |
| (For tableting processing) | | | | | |
| Flavor | 0.1 kg | 0.1 kg | 0.1 kg | 0.1 kg | 0.1 kg |
| Emulsifier | 2.8 kg | 2.8 kg | 2.8 kg | 2.8 kg | 2.8 kg |
| Vitamin C | 4.3 kg | 4.3 kg | 4.3 kg | 4.3 kg | 4.3 kg |
| Dextrin | — | 5.0 kg | — | — | 27.5 kg |
| Maltitol | 27.5 kg | — | — | 27.5 kg | — |
| total | 90.1 kg | 90.1 kg | 90.1 kg | 90.1 kg | 90.1 kg |

(1) Measurement of Hardness of N-Acetylglucosamine Tablet Disintegrating in Oral Cavity

For each N-acetylglucosamine tablet disintegrating in oral cavity obtained above, hardness was measured in accordance with a hardness measurement method established by Japanese Pharmacopoeia using a hardness meter (product name “FY-KD-20”, manufactured by Fuji Yakuhin Kikai Co., Ltd.”).

(2) Monitoring Evaluation of N-Acetylglucosamine Tablet Disintegrating in Oral Cavity

Twenty subjects were allowed to eat the N-acetylglucosamine tablets disintegrating in oral cavity and to judge whether each tablet was good or not in terms of ease of chewing when the tablet was chewed in oral cavity (disintegrating property), mouth feeling after the tablet was chewed in oral cavity (solubility), and overall ease for intake (overall judgment) (⊚: 16 or more subjects judged the tablet as good, o: 11 or more and less than 16 subjects judged the tablet as good, Δ: 6 or more and less than 11 subjects judged the tablet as good, X: less than 6 subjects judged the tablet as good).

(3) Evaluation of Oral Cavity Disintegration Time for N-Acetylglucosamine Tablet Disintegrating In Oral Cavity

Twenty subjects were allowed to eat the N-acetylglucosamine tablet disintegrating in oral cavity. Then, each of them voluntarily notified that he had realized disintegration of the tablet by chewing in oral cavity, to thereby determine the duration of time required to dissolve the tablet only with saliva in the mouth and to almost completely disintegrate the tablet (oral cavity disintegration time) The results are represented as means of 20 subjects for each tablet of Example or Comparative Examples.

Results of the above-mentioned items (1), (2), and (3) are collectively shown in Table 2.

TABLE 2

| | | Comparative | Comparative | Comparative | Comparative |
|---|---|---|---|---|---|
| | Example | Example 1 | Example 2 | Example 3 | Example 4 |
| Hardness (kgf) | 7.9 | 7.7 | 13.8 | 4.8 | 3.0 |
| Ease of chewing | ⊚ | Δ | Δ | ◯ | ◯ |
| (disintegrating property) | | | | | |
| Mouth feeling | ⊚ | ◯ | ◯ | ⊚ | Δ |
| (solubility) | | | | | |
| Overall ease for intake | ⊚ | Δ | Δ | ◯ | Δ |
| (overall judgment) | | | | | |
| Oral cavity disintegration | 32 | 121 | 167 | 103 | 123 |
| time (seconds) | | | | | |

Table 2 shows that the tablet of Example has a hardness of 7.9 kgf and is evaluated as good on ease of chewing when the tablet was chewed in oral cavity (disintegrating property), mouth feeling after the tablet was chewed in oral cavity (solubility), and overall ease for intake (overall judgment). On the other hand, the tablet of Comparative Example 1, which was prepared by subjecting a saccharide having high moldability to granulation processing and mixing a saccharide having low moldability in a tableting processing step, had a hardness almost equal (7.7 kgf) to that of the tablet of Example but was judged as hard in oral cavity, and the overall judgment was bad.

The tablet of Comparative Example 2, which was obtained by simultaneous granulation processing of a saccharide having high moldability and a saccharide having low moldability, had a hardness as high as 13.8 kgf and was judged as hard in oral cavity, and the overall judgment was bad. The tablet of Comparative Example 3, which was obtained without using a saccharide having low moldability, had a hardness as low as 4.8 kgf and was judged as slightly soft, but the overall judgment was good. The tablet of Comparative Example 4, which was obtained without using a saccharide having high moldability, had a hardness as low as 3.0 kgf and was judged as soft, but the overall judgment was bad because of low solubility.

The duration of time, which is required to dissolve the tablet only with saliva in the mouth and to almost completely disintegrate the tablet (oral cavity disintegration time), was found to be 103 to 123 seconds in the tablets of Comparative Examples, while the duration of time in the tablet of Example was found to be 32 seconds, which was significantly short.

Note that the subjects ingested one N-acetylglucosamine tablet disintegrating in oral cavity of Example (containing 1,000 mg of NAG)/day continuously for one month, but no one reported any difficulty and botheration in continuous ingestion.

(4) Packing of N-Acetylglucosamine Disintegrating in Oral Cavity with PTP

The N-acetylglucosamine tablet disintegrating in oral cavity of Example was packed with a PTP using a PTP packaging apparatus (product name “HM-135”, manufactured by Techno Jidouki Seisakusyo) in accordance with a conventional method. Packing with PTP was carried out smoothly without problems, and the PTP-packed N-acetylglucosamine tablet disintegrating in oral cavity kept its shape during delivery and storage as well as at the time of taking out from the PTP package.

### INDUSTRIAL APPLICABILITY

An N-acetylglucosamine tablet disintegrating in oral cavity of the present invention is suitable as a supplement, health food, etc.

1. An N-acetylglucosamine tablet disintegrating in oral cavity, comprising N-acetylglucosamine, wherein the tablet has a tablet hardness of 5 kgf to 20 kgf and a mass per tablet of 1,000 mg to 3,000 mg and wherein the tablet rapidly disintegrates and dissolves in the oral cavity.

2. An N-acetylglucosamine tablet disintegrating in oral cavity according to claim 1, wherein the tablet comprises 30% by mass to 90% by mass of N-acetylglucosamine.

3. An N-acetylglucosamine tablet disintegrating in oral cavity according to claim 1 or 2, wherein the tablet comprises 500 mg to 2,000 mg of N-acetylglucosamine per tablet.

4. An N-acetylglucosamine tablet disintegrating in oral cavity according to claim 1, further comprising a saccharide having low moldability and a saccharide having high moldability.

5. An N-acetylglucosamine tablet disintegrating in oral cavity according to claim 4, wherein the saccharide having low moldability is at least one selected from the group consisting of xylitol, mannitol, lactose, glucose, sucrose, dextrin, sucrose, fructose, xylose, lactulose, fructo-oligosaccharide, malto-oligosaccharide, galacto-oligosaccharide, erythritol, and lactitol, and wherein the saccharide having high moldability is at least one selected from the group consisting of maltitol, maltose, sorbitol, and reduced palatinose.

6. An N-acetylglucosamine tablet disintegrating in oral cavity according to claim 4, further comprising β-carotene and/or vitamin A.

7. An N-acetylglucosamine tablet disintegrating in oral cavity according to claim 6, wherein an amount of the β-carotene is 0.004% by mass to 0.4% by mass and an amount of the vitamin A is 0.0007% by mass to 0.07% by mass.

8. An N-acetylglucosamine tablet disintegrating in oral cavity according to claim 1, wherein the tablet is packed in a PTP.

9. A process for producing an N-acetylglucosamine tablet disintegrating in oral cavity, which rapidly disintegrates and dissolves in the oral cavity, characterized by comprising: a granulation step of mixing or spraying a saccharide having low moldability with or on N-acetylglucosamine to perform coating and/or granulation and a tablet compression step of mixing the granulated matter obtained in the granulation step with a saccharide having high moldability to perform compression.

10. A process for producing an N-acetylglucosamine tablet disintegrating in oral cavity according to claim 9, wherein the tablet has a tablet hardness of 5 kgf to 20 kgf and a mass per tablet of 1,000 mg to 3,000 mg.

11. A process for producing an N-acetylglucosamine tablet disintegrating in oral cavity according to claim 10, wherein the tablet contains 30 to 90% by mass of N-acetylglucosamine, 0.01% by mass to 49% by mass of the saccharide having low moldability, and 0.01% by mass to 49% by mass of the saccharide having high moldability.

12. A process for producing an N-acetylglucosamine tablet disintegrating in oral cavity according to claim 10, wherein the tablet contains 500 mg to 2,000 mg of N-acetylglucosamine per tablet.

13. A process for producing an N-acetylglucosamine tablet disintegrating in oral cavity according to claim 11, wherein the tablet contains 500 mg to 2,000 mg of N-acetylglucosamine per tablet.

14. A process for producing an N-acetylglucosamine tablet disintegrating in oral cavity according to any one of claims 9 to 13, comprising mixing β-carotene and/or vitamin A in the granulation step or the tablet compression step.
